(19) United States
(12) Patent Application Publication
(10) Pub. No.: US 2025/0073291 A1
(43) Pub. Date: Mar. 6, 2025

(54) HEMP EXTRACT-BASED COMPOSITIONS AND THEIR USES FOR IMPROVING MIND-BODY-BALANCE

(71) Applicant: Blaze n Chill, LLC, Mcminnville, OR (US)

(72) Inventor: Matthew Murray, Mcminnville, OR (US)

(21) Appl. No.: 18/823,935

(22) Filed: Sep. 4, 2024

Related U.S. Application Data
(60) Provisional application No. 63/580,456, filed on Sep. 5, 2023.

(51) Int. Cl. A61K 36/07; A61K 9/00; A61K 9/48; A61K 31/00; A61K 31/197; A61K 33/08; A61K 47/12; A61K 47/24; A61K 47/46; A61P 25/00

(52) U.S. Cl. CPC A61K 36/07; A61K 9/0095; A61K 9/4858; A61K 9/4875; A61K 31/197; A61K 31/658; A61K 33/08; A61K 36/3482; A61K 47/12; A61K 47/24; A61K 47/46; A61P 25/00

(57) ABSTRACT

The present disclosure relates to natural ingredient compositions comprising hemp extracts and other optional active ingredients; and, to methods of their use for mind-body-balance including for improved energy, gut health, focus, relaxation, and sleep.

### CROSS-REFERENCE TO RELATED APPLICATIONS

This application claims priority to U.S. provisional application No. 63/580,456 filed on Sep. 5, 2023 incorporated herein by reference in its entirety.

### BACKGROUND OF THE DISCLOSURE

Hemp extract-based compositions have great, unrealized potential to improve people's mind-body-balance. Demand for cannabinoid-based products continues to grow, but to-date there is limited innovation for them. There is a long felt need for more thoughtful, scientifically-designed hemp-based compositions for improving various facets of the daily human living experience that is met by the present disclosure.

### SUMMARY OF THE DISCLOSURE

The present disclosure relates to natural ingredient compositions comprising hemp extracts and methods of their use for mind-body-balance including for improved energy, gut health, focus, relaxation, and sleep.

The present disclosure provides hemp extract-based compositions that provide one or more of many benefits including but not limited to an improved general well-being, increasing energy levels, improving gut health, providing a greater ability to focus, promoting relaxation, boosting productivity, enhancing feelings of calm, providing pain relief, improving cognitive function, increasing ability to focus, enhancing clarity, and/or achieving better sleep patterns.

In some embodiments, the present disclosure provides healthy, beneficial compositions that normalize cannabinoids as an active ingredient in consumer products, but not being the only ingredient.

In some embodiments, the compositions of the present disclosure comprise various active ingredients that act in a synergistic manner to improve one or more aspects of mind-body-balance following their administration to an individual.

In some embodiments, the compositions of the present disclosure in addition to comprising active ingredients further comprise various inactive ingredient that enhance the experience of administering the compositions and products of the present disclosure and/or that help make the compositions and products of the present disclosure act faster or better to further improve one or more aspects of the mind-body-balance.

In some embodiments, the present disclosure provides cannabinoids combined with other specific healthy, active ingredients including but not limited to magnesium, Lion's mane mushroom (Hericium erinaceus) and matcha (Camelia sinensis).

In some embodiments, the compositions of the present disclosure are in forms including but not limited to pills, tablets, capsules, gummies, soft gels (aka softgels), liquids, tinctures, and powders.

In some embodiments, the compositions of the present disclosure can be provided as conventional food products, including but not limited to teas, bars, chips, crackers, and the like.

In some embodiments, the compositions of the present disclosure can be provided in specialty forms including but not limited to Jello shots, toothpicks, freeze pops, honey sticks, chewing gum, and packets of powder.

In some embodiments, the compositions of the present disclosure can be provided in drinks, wherein examples of such drinks include but are not limited to sodas, teas, juices, energy drinks, seltzers, and sparkling water, etc. and wherein the drinks can be any suitable packaging including but not limited to cans, boxes, bottles, jugs, and cartons.

In some embodiments, the present disclosure provides cannabinoids in consumer products that are enjoyed routinely including but not limited to tea bags, pack of chewing gum, and toothpicks either packaged individually or as a set. The toothpicks can be provided in cardboard or paper boxes/sleeves, tin boxes, toothpick dispensers, and the like.

In some embodiments, the present disclosure provides cannabinoid-based compositions that can assist with reducing the desire for or dependency on the consumption of including but not limited to caffeine, nicotine, betel leaf, coca leaf, and other potentially habit-inducing consumer products, as well as the unwanted and/or unhealthy effects of such other consumer products.

In some embodiments, the present disclosure provides hemp-extract based compositions that are designed to appeal to a cross-section of society, including but not limited to female consumers, millennial mothers, male and female millennial professionals, college and university sorority members, Gen Z males and females, and Gen X males and females, wherein some of these categories of individuals are not mutually-exclusive.

In some embodiments, the present disclosure provides hemp-extract based compositions that can be used for but not limited to the human situations of socializing, promoting healthier living, and leisure pleasure.

In some embodiments, the present disclosure provides hemp-extract based compositions that can be viewed as following into one of two major categories of human experiences: (1) increasing overall energy and focus; or (2) improving overall relaxation and sleep hygiene.

In some embodiments, the present disclosure provides compositions for oral administration comprising about 500 mg of Lion's mane mushroom (Hericium erinaceus) and a hemp extract standardized to deliver about 10 mg of cannabigerol (CBG), about 6 mg of delta-9-tetrahydrocannabivarin (THCV), and about 3 mg delta-9-tetrahydocannabinol (THC).

In some embodiments, the present disclosure provides compositions for oral administration comprising a hemp extract standardized to deliver about 10 mg cannabigerol (CBG) and about 6 mg of delta-9-tetrahydrocannabivarin (THCV).

In some embodiments, the present disclosure provides compositions for oral administration comprising a hemp extract standardized to deliver about 5 mg of delta-9-tetrahydrocannabivarin (THCV) and about 5 mg or about 10 mg delta-9-tetrahydocannabinol (THC).

In some embodiments, the present disclosure provides compositions for oral administration comprising a hemp extract standardized to deliver about 5 mg of cannabinol (CBN) and about 3 mg delta-9-tetrahydocannabinol (THC).

In some embodiments, the present disclosure provides such compositions for oral administration comprising about 300 mg of magnesium, about 125 mg L-Theanine, and a hemp extract standardized to deliver about 5 mg of cannabinol (CBN).

In some embodiments, the present disclosure provides such compositions wherein the magnesium is provided as magnesium gluconate and/or magnesium oxide.

In some embodiments. the present disclosure provides such compositions comprising one or more additional ingredients selected from the group consisting of a diluent, a flavoring, a preservative, a pH adjuster, a bitter blocker, and an emulsifier.

In some embodiments, the present disclosure provides such compositions wherein the diluent is water.

In some embodiments, the present disclosure provides such compositions wherein the one or more natural flavorings are selected from the group consisting of cinnamon, vanilla, monk fruit, peppermint, blueberry, citrus, chili, tropical fruit punch, and stevia.

In some embodiments, the present disclosure provides compositions wherein the one or more preservatives are selected from the group consisting of sodium benzoate and potassium sorbate.

In some embodiments. the present disclosure provides such compositions wherein the pH adjuster is citric acid.

In some embodiments, the present disclosure provides such compositions wherein the one or more emulsifiers are selected from the group consisting of VESIsorb® and sunflower lecithin.

In some embodiments, the present disclosure provides such compositions as a liquid.

In some embodiments, the present disclosure provides such compositions wherein the liquid is provided in a 60 mL dose.

In some embodiments, the present disclosure provides methods of administering the compositions disclosed herein to an individual, wherein the method comprises the individual orally ingesting the liquid compositions of the present disclosure.

In some embodiments, the present disclosure provides such compositions as soft gel capsule.

In some embodiments, the present disclosure provides methods of administering such compositions to an individual, wherein the method comprises the individual orally ingesting two soft gel capsules of the present disclosure.

In some embodiments, the present disclosure provides methods of improving an individual's mind-body-balance comprising administering any one of the compositions of the present disclosure to the individual.

### DETAILED DESCRIPTION OF THE DISCLOSURE

### I. Definitions

Unless stated otherwise, all technical and scientific terms used herein have the same meaning as commonly understood by those of ordinary skill in the art to which the disclosure belongs. While the following terms are believed to be well understood by one of ordinary skills in the art, the following definitions are set forth to facilitate explanation of the presently disclosed subject matter. Although any methods and materials similar or equivalent to those described herein can be used in the practice or testing of the present disclosure, preferred methods and materials are described. The following terms are defined below. These definitions are for illustrative purposes and are not intended to limit the common meaning in the art of the defined terms.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, “active,” “active ingredient,” and “other one or more active” all refer to one or more ingredients in a composition, formula, or product believed, known, or proven to exhibit advantageous properties when administered to an individual in need of improvement in one or more aspects of mind-body-balance as described herein.

As used herein, “additional ingredient,” “inactive ingredient,” or “other ingredient” all refer to those ingredients in a composition, formula, or product but not explicitly listed as “active” ingredients. Examples of such inactive ingredients include but are not limited to fillers, emulsifiers, lubricants, capsules, sweeteners, preservatives, and more. An inactive ingredient is not necessarily inert.

As used herein, “administration,” “administering,” or “to administer” all refer to the direct application of something (e.g., the compositions of the present disclosure) to the body of an individual, wherein the methods of application include but are not limited to injection, inhalation, ingestion, transdermal, inserted into the rectum or vagina, or by other means.

As used herein, “administered in combination with” or “co-administration” as used herein refers to the simultaneous or separate sequential administration in any manner of a solid or liquid oral dosage form containing the compositions, formulations, or products disclosed herein and one or more other active agents known to be useful for improving one or more aspects of mind-body-balance as described herein.

As used herein, “binder” refers to a substance or compound that promotes, provides, or improves cohesion, i.e., a substance that causes the components of a mixture to cohere to form a solid item that possesses integrity.

As used herein, “botanical” refers to a composition comprising a substance obtained from a plant (e.g., Cannabis extracts, Camelia extracts, Capsicum extracts).

As used herein, “carrier” refers to a substance that serves as a vehicle for improving the efficiency of delivery and the effectiveness of a pharmaceutical composition.

As used herein, “dietary ingredient” includes vitamins and minerals; herbs and other botanicals; amino acids; “dietary substances” that are part of the food supply, such as enzymes and live microbials (commonly referred to as “probiotics”); and concentrates, metabolites, constituents, extracts, or combinations of any dietary ingredient from the preceding categories.

As used herein, “composition” refers to the overall way that the parts (e.g., ingredients or formula) are put together.

As used herein, “dietary supplement” refers to a product intended for ingestion that, among other requirements, contains a “dietary ingredient” intended to supplement the diet.

As used herein, “disease” refers to a pathological process having a characteristic set of signs and symptoms. It may affect the whole body or any of its parts, and its etiology, pathology, and prognosis may be known or unknown.

As used herein, “drug” or “pharmaceutical” refer to a medicine or other substance labeled to treat or mitigate a disease.

As used herein, “excipient” refers to a inactive substance that is formulated in combination with an active ingredient of a composition and is inclusive of, but not limited to, disintegrants, lubricants, flavorings, bulking agents, binders, fillers, diluents, preservatives, antioxidants, and adjuvants, synergists and products used for facilitating drug absorption or solubility or for other pharmacokinetic considerations. See, also, The Handbook of Pharmaceutical Excipients, 4th edition, ed. by Rowe et al., American Pharmaceuticals Association (2003); and Remington: the Science and Practice of Pharmacy, 20th edition, Gennaro (ed.), Lippincott Williams & Wilkins (2003).

As used herein, “FDA” refers to the U.S. Food and Drug Administration.

As used herein, “food substance” refers to a food, food component, and/or dietary supplement ingredient.

As used herein, “formula” refers to a list of ingredients for or constituents of a composition according to the present disclosure.

As used herein, “full spectrum” or “full spectrum extract” refer to a plant extract having all or most of the chemical constituents in the plant have been preserved and included in the final product. As regards cannabis/hemp, full spectrum means that all or nearly all of the cannabinoids and terpenes found in the plant have been preserved and included in the final product.

As used herein, “health claim” refers to a relationship between a food substance, and a reduced risk of a disease or health-related condition.

As used herein, “hemp” refers to the 2018 U.S. Farm Bill definition of hemp as Cannabis sativa L. and “any part of that plant, including the seeds thereof and all derivatives, extracts, cannabinoids, isomers, acids, salts, and salts of isomers,” with no more than a 0.3 percent concentration of THC (i.e., ≤0.3% THC).

As used herein, “hemp extract” refers to a concentration of one or more components of the hemp plant's leaves, flowers, and stems. This extract can include cannabinoids, terpenes, plant sterols, fatty acids, flavonoids, and/or essential vitamins and nutrients. The extract may be refined to contain only one or more of these components.

As used herein, “herb” or “herbal” refer to a plant or plant part valued for its medicinal, savory, and/or aromatic qualities.

As used herein, “NF” refers to the National Formulary which includes standards for excipients, botanicals, and other similar products.

As used herein, “nutraceutical” refers to any product derived from food sources with extra health benefits in addition to the basic nutritional value found in foods.

As used herein, the term “mind-body-balance” refers generally to someone's personal wholeness and the feeling of well-being that occurs when their body, mind, and spirit are in harmony and balance. Changes in mind-body-balance can be based on one or both types of empirical data: qualitative data and quantitative data. Qualitative data can be categorized and involves descriptions that can be observed but not measured. Quantitative data is a measurement represented by a numerical value. Both types of empirical data can provide useful information in ascertaining changes in one's mind-body-balance before and after being administered the compositions, formulas, and products of the present disclosure.

As used herein, “nutrient content claim” refers to the level of a nutrient in a product, using terms such as free, high, and low, or compares the level of a nutrient in a food substance to another food substance, using terms such as more, reduced, and lite.

As used herein, “product” refers to anything a business produces and/or sells that solves a market problem or addresses a customer's need or desire. Examples of products disclosed herein include but are not limited to nutraceuticals, supplements, botanicals, and the like.

As used herein, “structure/function” refers to describing the role of a nutrient or dietary ingredient intended to affect the normal structure or function of the human body, for example, “calcium builds strong bones.” In some other contexts, “structure/function” refers to characterizing how a nutrient or dietary ingredient acts to maintain such structure or function, for example, “fiber maintains bowel regularity,” or “antioxidants maintain cell integrity.”

As used herein, “supplement” refers to concentrated sources of nutrients (i.e. mineral and vitamins) or other substances with a nutritional or physiological effect that are marketed in “dose” form (e.g. pills, tablets, capsules, liquids in measured doses).

As used herein, “treatment,” “to treat,” or “treating” refer to methods for obtaining beneficial or desired results for a patient, including clinical results.

### II. Chemical Constituents of Cannabis

Cannabis is a complex plant with over 400 chemical entities. Atakan Z., Cannabis, a complex plant: different compounds and different effects on individuals, (Dec. 2012) Ther Adv Psychopharmacol 2(6):241-54. Dried cannabis was found to contain a wide variety of compounds, including cannabinoids, terpenoids, flavonoids, hydrocarbons, fatty acids, phenols, and other miscellaneous classes of compounds and their metabolites. A comprehensive literature analyses and the chemical analyses of mass spectral signals identified up to 62 unique compounds in the cannabis extract attributable to 22 unique signals based on the corresponding molecular weights of the compounds or the corresponding fragments. Lewis et al., (2017) ACS Omega 2, 9, 6091-6103.

To date over 120 cannabinoids, the so-called phytocannabinoids, have been isolated from the cannabis plant. Morales et al., Molecular Targets of the Phytocannabinoids: A Complex Picture. Prog Chem Org Nat Prod. (2017) 103:103-131. The compositions, formulas, and products of the present disclosure comprise cannabinoids that come directly from a cannabis/hemp plant, i.e., natural cannabinoids with no modifications (i.e., no synthetic cannabinoids). The cannabinoids utilized in the compositions, formulas, and products of the present disclosure solely or primarily include the following: delta-9-tetrahydrocannabinol (Δ-9-tetrahydrocannabinol, THC D9) (aka THC D-9, THC Δ9, THC Δ-9, 49-THC, Δ⁹-THC, Delta9, Delta 9, and Delta-9), Δ-9-tetrahydrocannabivarin (THCv or THCV), cannabichromene (CBC), cannabidiol (CBD), cannabigerol (CBG), and cannabinol (CBN).

A more complete but not complete list of cannabinoids in cannabis/hemp includes the following: cannabicyclolic acid (CBLA), cannabicyclol (CBL), and cannabicyclovarin (CBLV), cannabichromene (CBC), cannabichromevarinic acid (CBCVA), cannabichromenic acid (CBCA), and cannabichromevarin (CBCV), cannabielsoin (CBE), cannabielsoin acid A (CBEA-A), and cannabielsoic acid B (CBEA-B), 10-Ethoxy-9-hydroxy-delta-6a-tetrahydrocannabinol, 8,9-Dihydroxy-delta-6a-tetrahydrocannabinol, cannabitriol (CBT), and cannabitriolvarin (CBTV), cannabidiol (CBD), cannabidiol monomethylether (CBDM), cannabidiolic acid (CBDA), cannabidivarinic acid (CBDVA), cannabidiorcol (CBD-C1), cannabidivarin (CBDV), and cannabidiphorol (CBDP), cannabigerol (CBG), cannabigerovarin (CBGV), cannabigerovarinic acid (CBGVA), cannabigerol monomethylether (CBGM), cannabigerolic acid monomethylether (CBGAM), and cannabigerolic acid (CBGA), cannabinolic acid (CBNA), cannabinodiol (CBND), cannabinodivarin (CBVD), cannabinol (CBN), cannabiorcool (CBN-C1), cannabivarin (CBV), cannabinol methylether (CBNM), cannabinol-C2 (CBN-C2), and cannabinol-C4 (CBN-C4), delta-8-tetrahydrocannabinol (Δ8-THC) and delta-8-tetrahydrocannabinolic acid (Δ8-THCA), delta-9-tetrahydrocannabinol (THC), Delta-9-tetrahydrocannabinol-C4 (THC-C4), delta-9-tetrahydrocannabiorcol (THC-C1), delta-9-tetrahydrocannabiorcolic acid (THCA-C1), delta-9-tetrahydrocannabinolic acid-C4 (THCA-C4), delta-9-tetrahydrocannabivarin (THCV), delta-9-tetrahydrocannabivarinic acid (THCVA), delta-9-tetrahydrocannabinolic acid A (THCA-A), delta-9-tetrahydrocannabinolic acid B (THCA-B), and delta-9-tetrahydrocannabiphorol (THCP), 10-Oxo-delta-6a-tetrahydrocannabinol (OTHC), cannabichromanon (CBCF), cannabifuran (CBF), cannabiglendol, Delta-9-cis-tetrahydrocannabinol (cis-THC), cannbicitran (CBT), dehydrocannabifuran (DCBF), tryhydroxy-delta-9-tetrahydrocannabinol (triOH-THC), and cannabiripsol (CBR). For a comprehensive summary of the cannabinoids in cannabis see, e.g., Chief Botanicals, Complete List of Cannabinoids in Cannabis (2013).

The 10 most common cannabinoids found in products today include delta-9-Tetrahydrocannabinol (THC), delta-8-Tetrahydrocannabinol (D8THC), cannabidiol (CBD), cannabinol (CBN), cannabichromene (CBC), cannabigerol (CBG), tetrahydrocannabinolic acid (THCA), cannabidiolic Acid (CBDA), tetrahydrocannabivarin (THCV or THCv), and cannabidivarin (CBDV).

Δ⁹-tetrahydrocannabinol (aka Δ⁹-THC or Delta-9) is also known by its International Non-Proprietary Name (INN) as dronabinol. The unsaturated bond in the cyclohexene ring is located between C-9 and C-10 in the more common dibenzopyran ring numbering system. There are four stercoisomers of THC, but only the (-)-trans isomer occurs naturally (CAS-1972-08-3). The fully systematic name for this THC isomer is (-)-(6aR, 10aR)-6,6,9-trimethyl-3-pentyl-6a,7,8,10a-tetrahydro-6H-benzo[c]chromen-1-ol. Two related substances, Δ⁹-tetrahydrocannabinol-2-oic acid and Δ⁹-tetrahydrocannabinol-4-oic acid (THCA), are also present in cannabis, sometimes in large amounts. The active isomer Δ⁸-THC, in which the unsaturated bond in the cyclohexene ring is located between C-8 and C-9, is found in much smaller amounts. Cannabis Drug Profile, European Monitoring Centre for Drugs and Drug Addiction (2023) online website.

The cannabis plant has more than 200 terpenes, many of which are only present in trace amounts. They are the chemical building blocks of essential oils in plants. Cannabis plants produce terpenes along with cannabinoids in glands called trichomes. Besides giving cannabis its taste and aroma, some terpenoids are believed to also have beneficial effects. The most prominent terpenes in cannabis plants include but are not limited to myrcene, beta-caryophyllene (aka beta caryophyllene and β-caryophyllene), limonene, linalool, pinene, humulene, terpinolene, alpha-bisabolol, eucalyptol, geraniol, terpineol, farnesene, borneol, ocimene, nerolidol, guaiol, valencene, delta-3 carene, phytol, sabinene, phellandrene, fenchol, menthol, terpinene, isoborneol, and cymene. Other possible terpenes that might be present include but are not limited to octanol, isopulegol, cedrene, camphene, geranyl acetate, bergamotene, camphor, and pulegone. Terpenes are simple hydrocarbons, while terpenoids are a modified class of terpenes with different functional groups and having an oxidized methyl group moved or removed at various positions. For a complete list of cannabis-derived terpenes see, e.g., Liz G., The Complete List of Cannabis-Derived Terpenes (Jan. 2, 2021) Cannabis Trends, Cannabinoids.

For additional information on the chemical constituents of cannabis see, e.g., Armentano, et al., Clinical Applications for Cannabis & Cannabinoids: A Review of the Recent Scientific Literature (Oct. 31, 2021), NORML, 138 pages; Backes et al., Cannabis Pharmacy: The Practical Guide to Medical Marijuana-Revised and Updated (Nov. 14, 2017) Black Dog & Leventhal, New Edition, 320 pages; Thomas et al., The Analytical Chemistry of Cannabis: Quality Assessment, Assurance, and Regulation of Medicinal Marijuana and Cannabinoid Preparations (Emerging Issues in Analytical Chemistry) Elsevier, 1st Edition, 132 pages; and Kinghorn, Phytocannabinoids: Unraveling the Complex Chemistry and Pharmacology of Cannabis sativa (Progress in the Chemistry of Organic Natural Products Book 103) Springer, 1^{st} Edition, 140 pages.

Botanical cannabinoids and purified CBD show relatively low toxicity and lack any dependence profile while having a broad spectrum of therapeutic properties, such as anticonvulsive, anxiolytic, neuroprotective, antidepressant, anti-inflammatory, and immunomodulating activities (Russo et al 2006). Epidiolex® is presently the only FDA approved CBD pharmaceutical. According to its FDA Label, Epidiolex® is an oral solution indicated for the treatment of seizures associated with Lennox-Gastaut syndrome or Dravet's syndrome in patients 2 years of age and older.

### III. Extracts of Hemp and Other Botanicals

In some embodiments, the present disclosure provides for extracts and compositions developed or derived from hemp plants as disclosed herein. In other embodiments, said methods can be used with any Cannabis plants. In some embodiments, the extracts and of the present disclosure are full spectrum hemp extracts or comprise full spectrum hemp extracts. In some embodiments, the extracts used in the present disclosure are extracted via methods that preserve the cannabinoid and terpenes. In some embodiments, the extracts comprise specific chemical constituents or subsets of chemical constituents. In some embodiments, the specific chemical constituents or subsets of chemical constituents are standardized to an amount to be provided as a dose or part of a dosing schedule to deliver certain amounts over certain periods of time.

In some embodiments, the hemp extracts are purchased commercially and/or provided by third party producers to be used in the compositions and formulas of the present disclosure. There are hundreds of hemp extract providers offering a multitude of hemp extract products which could be utilized to make the compositions and formulas of the present disclosure.

The hemp extracts utilized herein regardless of how they are obtained are designed to be used in the compositions, formulas, and products of the present disclosure for human or animal consumption via administration methods including but not limited to inhalation (via combustion, vaporization, and nebulization), buccal absorption within the mouth, oral administration (e.g., eating/drinking), and topical application delivery methods.

Cannabis Oils and Co₂ Extraction. Solvent reduced oils are also sometimes referred to as an oil, butane hash oil (BHO), CO₂extract, among other names. This type of extract is made by soaking plant material in a chemical solvent capable of solubilizing one or more chemical constituents of the plant (e.g., cannabinoids and/or terpenes). After separating the solvent from plant material, the solvent can be boiled or evaporated off, leaving the extract “oil” behind Butane Hash Oil is produced by passing butane over cannabis and then letting the butane evaporate. Rick Simpson Oil is produced through isopropyl, or ethanol extraction of cannabis. The resulting substance is a wax like golden brown paste. Another common extraction solvent for creating Cannabis oil is CO₂. Persons having skill in the art will be familiar with CO₂ extraction techniques and devices, including those disclosed in US Published Patent Application Nos. U.S. 20160279183, US 2015/01505455, and US 2018/0000857: U.S. Pat. Nos. 9,730,911, 9,826,689, 9,867,859, 9,919,241, 10,307,446, 10,308,625, 10,315,129, 10,406,186, 10,570,350, 10,688,410, 11,118,131, 11,241,468, 11,291,699, and 11,541,089; and International Published Patent Applications WO 2004/016277 and WO 2021/084527.

Heat extractions. The present disclosure also teaches the use of extracts produced via heat-based extraction methods, such as those disclosed in US Patent Application Nos. US 2018/0078874, US 2019/0151771, US 2019/0076753, and U.S. Pat. No. 10,159,908, each of which is hereby incorporated by reference for all purposes. In some embodiments, the plants of the present disclosure can be extracted by exposing tissue to a hot air gas stream that volatizes cannabinoids and/or other secondary metabolites of the plant, which are then condensed and recovered in tanks.

In some embodiments, the present disclosure teaches the use of extracts produced by exposing plants, plant parts or plant cells to vaporizing heat. As used herein, the term “vaporizing heat” refers to heat sufficient to volatize one or more terpene on cannabinoid components of said plant, plant part or plant cell. The boiling points for each of the cannabinoid and terpene constituents of a hemp plant are well known or readily ascertainable. In some embodiments, vaporizing heat comprises 150° F., 155° F., 160° F., 165° F., 170° F., 175° F., 180° F., 185° F., 190° F., 195° F., 200° F., 205° F., 210° F., 215° F., 220° F., 225° F., 230° F., 235° F., 240° F., 245° F., 250° F., 255° F., 260° F., 265° F., 270° F., 275° F., 280° F., 285° F., 290° F., 295° F., 300° F., 305° F., 310° F., 315° F., 320° F., 325° F., 330° F., 335° F., 340° F., 345° F., or 350° F., and all ranges and subranges therebetween.

Tinctures. Tinctures are alcoholic extracts of Cannabis. These are usually made by mixing Cannabis material with high proof ethanol and separating out plant material. Within the dietary supplement industry “tincture” may also describe an oil dilution of hemp extract.

Chemical Extraction. The chemical extraction of specialty Cannabis can be accomplished employing polar and non-polar solvents in various phases at varying pressures and temperatures to extract terpenes, cannabinoids, and other compounds of flavor, fragrance, or pharmacological value selectively or comprehensively for use individually or combination in the formulation of our products. The solvents employed for selective extraction of our cultivars may include water, carbon dioxide, 1,1,1,2-tetrafluoroethane, butane, propane, ethanol, isopropyl alcohol, hexane, and limonene, in combination or series. It is also possible to extract compounds of interest mechanically by sieving the plant parts that produce those compounds. Measuring the plant part, i.e. trichome gland head, to be sieved via optical or electron microscopy can aid the selection of the optimal sieve pore size, ranging from 30 to 130 microns, to capture the plant part of interest. The chemical and mechanical extraction methods of the present disclosure can be used to produce products that combine chemical extractions with plant parts containing compounds of interest.

The hemp extracts as used in the present disclosure may also be combined with pure compounds of interest to the extractions, e.g. cannabinoids or terpenes to further enhance or modify the resulting formulation's fragrance, flavor, or pharmacology. In some embodiments, extracts from the hemp lines of the present disclosure are combined with one or more additional active or inactive compounds as disclosed herein. In some embodiments, extracts of the present disclosure, such as whole hemp extracts, or a purified cannabinoid from said hemp plant, can be combined with another cannabinoid or terpene to produce a composition.

### IV. Active Ingredients

The hemp-based extracts utilized in some of the compositions, formulas, and products of the present disclosure contain one or more active compounds including but not limited to THC (e.g., Delta9-THC), CBD, THCV, CBG, CBN, terpenes, and terpenoids. The preparation of hemp-based extracts and the active constituents of such hemp-based extracts are described above and elsewhere herein.

Non-hemp based active compounds utilized in some of the compositions, formulas, and products of the present disclosure include but are not limited to Lion's Mane Mushroom (Hericium erinaceus), magnesium (e.g., magnesium gluconate, magnesium oxide), L-Theanine, and Matcha Powder (Camelia sinensis).

Lion's mane mushroom (Hericium erinaceus) (aka Monkey's head mushroom, mountain-priest mushroom, bearded tooth fungus, and bearded hedgehog) is an edible mushroom that is well-established as a culinary and medicinal mushroom for brain and nerve health. Lion's mane mushrooms, and its powders, extracts, tinctures, etc. are readily available from numerous third parties and commercial sources. Lion's mane mushroom compositions and methods for making and using Lion's mane mushrooms are well documented in the literature. See, e.g., International Publication Number WO 2011/094579; Japanese Patent JP4965801; Japanese Patent JP2006117541; Japanese Patent JP2003212790; Lai et al., Neurotrophic properties of the Lion's mane medicinal mushroom, Hericium erinaceus (Higher Basidiomycetes) from Malaysia (2013) Int J Med Mushrooms 15(6):539-5; Kushairi et al. Lion's Mane Mushroom, Hericium erinaceus (Bull.: Fr.) Pers. Suppresses H₂O₂-Induced Oxidative Damage and LPS-Induced Inflammation in HT22 Hippocampal Neurons and BV2 Microglia (Aug. 2019) Antioxidants (Basel) 1;8(8):261; Mendel Friedman, Chemistry, Nutrition, and Health-Promoting Properties of Hericium erinaceus (Lion's Mane) Mushroom Fruiting Bodies and Mycelia and Their Bioactive Compounds (2015) J. Agric. Food Chem. 63, 32, 7108-7123; and, Syed Asim Shah Bacha et al., Lion's mane mushroom; new addition to food and natural bounty for human wellness: A review (Oct 2018) International Journal of Biosciences Vol. 13, No. 4: 396-402.

Magnesium is a chemical element with the symbol Mg and atomic number 12. Magnesium is a key factor in making several parts of the body run smoothly: the heart, bones, muscles, nerves, and others. Without enough magnesium, these areas malfunction. This is summarized in research, which finds that a magnesium deficiency or low magnesium diet leads to health problems. There are many types of magnesium present in dietary supplements and food products, including but not limited to magnesium citrate, magnesium glycinate, magnesium chloride, magnesium lactate, magnesium malate, magnesium taurate, magnesium sulfate, and magnesium oxide. Each type of magnesium has different properties. They can vary in terms of their medical uses, bioavailability, and potential side effects. Magnesium gluconate is a mineral supplement which is used to prevent and treat low levels of magnesium. These magnesium forms, including magnesium gluconate, are widely and readily available from numerous third parties and commercial sources.

Theanine is an amino acid found in tea and some mushrooms. It comes in two forms: L-theanine and D-theanine. Theanine is used to improve brain function and for anxiety, mental impairment, stress, and other conditions. Theanine, including L-theanine, is widely and readily available from numerous third parties and commercial sources.

Matcha Powder (Camelia sinensis) is finely ground powder of specially grown and processed green tea leaves. Matcha is popular in health stores and coffee shops, available as matcha shots, lattes, teas, and desserts. Farmers shade the plants used for matcha for most of the growth period which increases chlorophyll production, boosts the amino acid content, and gives the plant a darker green hue. Matcha is rich in catechins, a class of plant compounds in tea that act as natural antioxidants. Consumption of matcha and its components is believed to help protect the liver, promote heart health, and even aid in weight loss. Matcha powder is widely and readily available from numerous third parties and commercial sources.

### V. Additional Ingredients

In some embodiments, the hemp extract-based compositions, formulas, and products of the present disclosure comprise one or more additional botanical extracts, wherein such additional extracts can be in the form of including but not limited to one or more oils and/or oil-like substances. Examples of suitable oils include but are not limited to glyceryl monolinoleate, corn oil, olive oil, sesame oil, soybean oil (e.g., hydrogenated soybean oil), rapeseed oil, coconut oil, sunflower oil, and combinations thereof. In some embodiments, the oils are purified oils and/or highly refined oils. Examples of specific suitable oils include but are not limited to Refined Olive Oil IV, which can be used as an excipient or API in the formulation of poorly soluble drugs, injectables, syrups, and parenteral nutrition for human and veterinary applications); Refined Sesame Oil IV-1, which can be used as an excipient in the formulation of lipophilic drugs, injectables, and syrups for human and veterinary applications; and Refined Soybean Oil IV, which can be used as an excipient or API in the formulation of poorly soluble drugs, injectables, syrups, and parenteral nutrition for human and veterinary applications. All three of these specialty oils are available from ADM®.

In some embodiments, the compositions, formulas, and products of the present disclosure comprise or consist of constituents or ingredients that are listed on the Food and Drug Administration (FDA) Generally Regarded as Safe (GRAS) database.

In some embodiments, the compositions, formulas, and products of the present disclosure comprise or consist of constituents or ingredients that are compliant with the standards established by the NF.

The compositions, formulas, and products of the present disclosure are to be manufactured in accordance with current good manufacturing practices (cGMPs).

In some embodiments, the purified oils are also used as excipients such as during the formulation of soft gel capsules, syrups, and lotions.

In some embodiments, the compositions, formulas, and products disclosed herein may also comprise other conventional acceptable food, supplement, and/or nutraceutical ingredients, commonly referred to as carriers, excipients, or adjuvants, excipients or adjuvants including but not limited to: disintegrants, binders, lubricants, glidants, stabilizers, fillers, diluents, colorants, flavoring agents, and preservatives. For example, useful additives include materials such as agents for retarding dissolution (e.g., paraffin), resorption accelerators (e.g., quaternary ammonium compounds), surface active agents (e.g., cetyl alcohol, glycerol monostearate, and sodium lauryl sulfate), adsorptive carriers (e.g., kaolin and bentonite), preservatives, sweeteners, coloring agents, flavoring agents (e.g., chocolate mint, citric acid, menthol, glycine or orange powder), stabilizers (e.g., citric acid or sodium citrate), binders (e.g., hydroxypropylmethylcellulose), and mixtures thereof. Those of ordinary skill in the art may, by conventional experimentation, select one or more of the above carriers based on the desired properties of the dosage form without undue burden. The amount of each carrier used is within the conventional range in the art.

The term “carrier” as defined above and used herein includes any and all solvents, dispersion media, coating agents, surfactants, antioxidants, preservatives (e.g., antibacterial agents or antifungal agents), isotonic agents, absorption delaying agents, salts, preservatives, drugs, drug stabilizers, binders, excipients, disintegrants, lubricants, sweeteners, flavoring agents, dyes, and the like, and combinations thereof, as known to those skilled in the art (see, e.g., Remington's Pharmaceutical Sciences, 18th edition, Mack Printing Company, 1990, 1289-1329). The use of any conventional carrier in therapeutic, pharmaceutical, food, supplement, and nutraceutical compositions is contemplated herein unless it is incompatible with the active ingredient(s) of the present disclosure.

In some embodiments, typical binders that can be used for formulating dosage forms of the present disclosure include but are not limited to natural, synthetic, or semi-synthetic glycerides, hydrogenated coco-glycerides, mineral oil, gelatins, starches, sucrose and sucrose derivatives, lactose and lactose derivatives, cellulose, methyl cellulose, microcrystalline cellulose (e.g., AVICEL PH from FMC (Philadelphia, Pa.), hydroxypropyl cellulose, hydroxyethyl cellulose, hydroxypropyl methyl cellulose METHOCEL from Dow Chemical Corp., Midland, Mich.) and other cellulose derivatives, sucrose; dextrose; corn syrup; polysaccharide; gelatin, silicones (e.g., dimethicone, bis-vinyl dimethicone/dimethicone copolymer), isopropyl myristate, isostearyl palmitate, polyvinylpyrrolidone and derivatives, and polyglycols, (e.g., polyethylene glycols (PEGs), polyethylene oxides (POE), methoxpolyethylene glycols, polypropylene glycols, polybutylene glycols or derivatives thereof having a molecular weight that is sufficient to provide the necessary hardness and time for dissolution of the dosage form). Non-limiting examples of some specific polyglycol derivatives that can be used are: (a) PEG-laureates and dilaureates (e.g., PEG-10-, PEG-12-, PEG-20-, PEG-32-laurates, PEG-20- and PEG-32-dilaurates, PEG-20-glyceryl-, PEG-30-glyceryl- and PEG-40-glyceryl-laurates, PEG-80-sorbitan laurate); (b) PEG-oleates, dioleates and trioleates (e.g., PEG-12-, PEG-15-, PEG-20-, PEG-32, PEG-200- and PEG-400-olcates, PEG-20- and PEG-32-dioleates, PEG-20-trioleate, PEG-25-glyceryl trioleate, PEG-20-glyceryl- and PEG-30-glyceryl-oleates, PEG-40-sorbitan oleate); (c) PEG-stearates and distearates (e.g., PEG-15-, PEG-40-, PEG-100-stearates, PEG-32-distearate and PEG-20-glyceryl stearate) (d) castor, palm kernel, corn and soya oil derivatives of PEG (e.g., PEG-35-, PEG-40- and PEG-60-castor oils, PEG-40-, PEG-50- and PEG-60-hydrogenated castor oils, PEG-40-palm kernel oil, PEG-60-corn oil, PEG-30-soya sterol); (e) other PEG derivatives (e.g., PEG-24- and PEG-30-cholesterol, PEG-25-phytosterol, PEG-6-and PEG-8-caprate/caprylate glycerides, tocopheryl PEG-100 succinate, PEG-15-100 octylphenol products and PEG-10-100 nonylphenol products); (f) other products such as polyglyceryl-10-laurate, POE-9-and POE-23-lauryl ethers, POE-10- and POE-20-oleyl ethers, POE-20-stearyl ether, polysorbate-20 (Tween 20), polysorbate-80 (Tween 80), polyglyceryl-10-oleate, Tween 40, Tween 60, sucrose monostearate, monolaurate, monopalmitate, and various products of Poloxamer series.

In some embodiments, typical excipients that can be used for formulating dosage forms of the present disclosure include but are not limited to gelatin, sodium saccharin, mannitol, stevioside, peppermint oil, cherry flavor, lemon oil, and raspberry flavor.

In some embodiments, the dosage forms of the present disclosure may optionally be formulated to further comprise one or several antioxidants. Examples of pharmaceutically acceptable antioxidants include but are not limited to: (1) water-soluble antioxidants, such as ascorbic acid, cysteine hydrochloride, sodium bisulfate, sodium metabisulfite and sodium sulfite; (2) oil-soluble antioxidants, such as ascorbyl palmitate, butylated hydroxyanisole (BHA), butylated hydroxytoluene (BHT), lecithin, propyl gallate and a tocopherol; and (3) metal chelating agents, such as citric acid, ethylenediamine tetraacetic acid (EDTA), sorbitol, tartaric acid and phosphoric acid. Additional examples of oxidants that could be used according to the present disclosure include but are not limited to α-tocopherol acetate, acetone sodium bisulfite, acetylcysteine, cysteine, tocopherol natural, tocopherol synthetic, dithiothreitol, monothioglycerol, nordihydroguaiarctic acid, propyl gallate, sodium formaldehyde sulfoxylate, sodium metabisulfite, sodium sulfite, sodium thiosulfate, thiourca and tocopherols.

In some embodiments, the dosage forms of the present disclosure may optionally be formulated to further comprise one or several adjuvants or synergists. If adjuvants or synergists are used, non-limiting examples of those that can be used include citric acid, EDTA (ethylenediaminetetraacetate) and salts, hydroxyquinoline sulfate, phosphoric acid, and tartaric acid.

In some embodiments, the dosage forms of the present disclosure may optionally further comprise one or several preservatives. If preservatives are used, non-limiting examples of those that can be used include benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, o-phenylethyl alcohol, and thimerosal.

Examples of acceptable surfactants for use in the present disclosure include but are not limited to polyvinylpyrrolidone, polyethylene glycol surfactants, oleic acid, and lecithin.

Examples of acceptable lubricants and glidants include, but are not limited to: silica gel, magnesium trisilicate, starch, talc, tricalcium phosphate, magnesium stearate, aluminum stearate, calcium stearate, magnesium carbonate, magnesium oxide, polyethylene glycol, powdered cellulose, and microcrystalline cellulose.

An example of an acceptable bitter blocker is ClearIQ™, a natural, clean-label bitter blocker and flavor clarifier made by Myco Technology™.

Examples of acceptable fillers and pharmaceutically acceptable diluents include, but are not limited to: powdered sugar, compressible sugar, glucose binding agents, dextrin, dextrose, lactose, mannitol, microcrystalline cellulose, powdered cellulose, sorbitol, sucrose, and talc.

In some embodiments, the hemp extract-based compositions, formulas, and products of the present disclosure comprise one or more natural or artificial flavoring agents including but not limited to orange flavoring, citric acid flavoring, cherry flavoring, cinnamon flavoring, mango flavoring, stevia flavoring, monk fruit flavoring, sucralose flavoring, strawberry flavoring, chocolate flavoring, mint flavoring (e.g., spearmint, peppermint), and combinations thereof (e.g., chocolate orange flavoring). The flavorings may be in any suitable format such as but not limited to oils, tinctures, powders, etc.

In some embodiments, the dosage forms of the present disclosure can be formulated, as appropriate, to include disintegrants, including but not limited to starch, cellulose derivatives and alginates, crosslinked sodium carboxymethyl cellulose (corscarmellose sodium) (e.g., AC-DI-SOL from FMC), hydroxypropylmethyl cellulose (HPMC), crosslinked polyvinylpyrrolidone (crospovidone), clay, cellulose, gum, crosslinked polymers (e.g., crospolyvinylpyrrolidone or crospovidone, such as POLYPLASDONE XL from ISP (International Specialty Products, Wayne, N.J.)), croscarmellose calcium, soybean polysaccharide, and guar gum.

The dosage forms of the present disclosure can be formulated, as appropriate, to include glidants, including but not limited to silicon dioxide, colloidal anhydrous silicon, and other silica compounds, and/or and or lubricants including stearic acid and salts thereof, such as magnesium stearate.

In some embodiments, the botanical extracts of the present disclosure may be a powdered botanical extract which may optionally be combined with one or more inactive, neutral compounds/ingredients which can be pharmaceutically acceptable excipients or carriers, including, but not limited to, binders, antioxidants, adjuvants, synergists and/or preservatives. In some embodiments, the botanical extract preparations of the present disclosure can further include encapsulating agents known in the art (i.e., phospholipids, cyclodextrins, etc.) to encapsulate the actives. These encapsulating agents can be employed with or without the use of aqueous or organic solvents. In some embodiments, the powdered botanical extract preparations may optionally be combined with one or more additional pharmaceutically active components.

In some embodiments, where the powdered botanical extracts of the present disclosure are optionally combined with one or more inactive, neutral ingredients or one or more additional pharmaceutically active components, the optional ingredients may be added during the process of preparing the powdered preparation, for example, while the mixture is still in its liquid form and before the solid product has been obtained. Alternatively, the optional ingredients may be added to the powdered preparation after the process of preparing the powdered preparation has been completed, and before further formulation into suitable dosage forms. In some embodiments, the powdered botanical extract of the present disclosure can be further incorporated into different dosage forms in accordance with formulation processes as are known in the art.

Glycerol monolinoleate (C₂₁H₃₈O₄; MW 354.5 g/mol; PubChem CID 5283469; CAS RN®: 26545-74-4) is a 1-monoglyceride that has octadecadienoyl (linoleoyl) as the acyl group. It is functionally related to linoleic acid. Glycerol monolinoleate is a mixture of monoglycerides, mainly glyceryl monooleate and glyceryl monolinoleate, together with variable quantities of diglycerides and triglycerides. Synonyms include 1-linoleoyl-fac-glycerol, monolinolein, 1-monolinolcin, and glycerol 1-monolinolato. Glyceryl monolinoleate is a natural product found in Saposhnikovia divaricate, Hycoscyamus niger, and other organisms. It can be used to enhance absorption of a substance, thereby enhancing bioavailability for systemic exposure. For additional information see USPNF Docid: GUID-B60F2346-5660-41F4-97FE-7721AECF2902_2_en-US.

In some embodiments of the present disclosure, the compositions of the present disclosure can be stored under US Pharmacopeia (USP) controlled room temperature conditions of about 20° C. to about 25° C. (about 68° F. to about 77° F.) with excursions permitted between about 15° C. to about 30° C. (about 59° F. to about 86° F.). In some embodiments, the compositions and products of the present disclosure can be stored at room temperature. While room temperature is defined differently in different places, it generally refers to a range somewhere between about 68 degrees Fahrenheit and about 74 degrees Fahrenheit. The compositions and products of the present disclosure are expected to store at room temperatures for up to about 18 months. Stability tests are being conducted to obtain a more exact timeframe for storage.

### VI. Administration Forms and Routes

The compositions and products of the present disclosure encompass many forms. In some embodiments, the present disclosure provides hemp oils and tinctures to be used in the compositions of the present disclosure. In some embodiments, the present disclosure provides hemp extract-based liquids, capsules, or soft gels.

In some embodiments of the present disclosure, the route of administration for the compositions of the present disclosure is oral without further dilution or reconstitution.

In some embodiments, the hemp extract-based compositions, formulas, and products of the present disclosure can be formulated in many different forms and finished formats including but not limited to emulsifications, particle encapsulations, powders, liquids, solids, etc.

In some embodiments, the hemp extract-based compositions, formulas, and products of the present disclosure are administered orally, wherein such oral administration may be in a form including but not limited to capsules, strips, tablets, pills, tinctures, sprays, lozenges, liquids, soft gels, etc.

In some embodiments, pharmaceutical compositions of the present disclosure may be suitable for delivery to the respiratory tract using a metered dose inhaler (MDI). Polar excipients are used routinely in pharmaceutical compositions for treating respiratory disorders that are delivered using MDIs. They are also sometimes referred to as solvents, co-solvents, carrier solvents and adjuvants. Their inclusion can serve to solubilize a surfactant or the drug in the propellant and/or inhibit deposition of drug particles on the surfaces of the metered dose inhaler that are contacted by the pharmaceutical composition as it passes from the container in which it is stored to the nozzle outlet. They are also used as bulking agents in two-stage filling processes where the drug is mixed with a suitable polar excipient. The most used polar excipient is ethanol. If a polar excipient is used, it will typically be present in an amount of from 0.5 to 10% by weight, preferably in an amount of from 1 to 5% by weight based on the total weight of the pharmaceutical composition.

Some embodiments of the present disclosure are directed to dosage forms that are formulated as solid articles suitable for sublingual or oral administration, such as troches, lozenges, pills, oral dissolving strips, caps, or boluses. These solid dosage forms typically comprise additional excipients. They can be prepared by first mixing the powdered botanical extract of the present disclosure with one or more suitable excipients followed by molding or compressing the blended mixture. Both hard and chewable lozenges and troches are within the scope of the present disclosure. In some embodiments, the oral dosage forms are formulated as capsules in which the powdered botanical extracts are encapsulated in soft or hard gelatin capsules.

In some embodiments, the compositions of the present disclosure are directed to dosage forms that are formulated as topical formulations, including but not limited to creams, ointments, and gel, using formulation methods as are known in the art. In one embodiment, the topical formulation is a transdermal patch, using formulation methods and technologies as are known in the art.

In some embodiments, the compositions of the present disclosure are directed to dosage forms that are formulated as solid articles suitable for administration as vaginal ovules or rectal suppositories, using formulation methods as are known in the art.

Some embodiments of the present disclosure are directed to dosage forms that are formulated as liquids, including but not limited to emulsions, liposomes, dispersions, oils, and tinctures, using formulation methods as are known in the art.

Some embodiments of the present disclosure are directed to dosage forms that are formulated as beverages and edibles, wherein the powdered botanical extract is incorporated into food and drink products.

Some embodiments of the present disclosure are directed to dosage forms that are formulated as smokeable or vaporizable formulations.

The optimal dose of each combination partner for administration can be determined empirically for everyone using known methods and will depend upon a variety of factors, including, but not limited to: the degree of progression of the condition; the age, body weight, general health, gender, and diet of the individual; the time and route of administration; and other medications the individual is taking. Optimal doses may be established using routine testing and procedures that are well known in the art.

The amount of each combination partner that may be combined with the carrier materials to produce a single dosage form will vary depending upon the individual treated and the mode of administration. In some embodiments, the unit dosage forms containing the combination of agents as described herein will contain a certain amount of each agent of the combination that is typically administered when the agent is administered alone.

### VII. Dosages and Dosing

The hemp extract dosing regimens of the serving sizes of the present disclosure include standardized dosages of CBG of about 5 mg, about 6 mg, about 7 mg, about 8 mg, about 8.5 mg, about 9 mg, about 9.5 mg, about 9.75 mg, about 9.8 mg, about 9.85 mg, about 9.9 mg, about 10 mg, about 10.1 mg, about 10.15 mg, about 10.2 mg, about 10.25 mg, about 10.5 mg, about 11 mg, about 12 mg, about 13 mg, about 14 mg, and about 15 mg.

In some embodiments, the CBG dosing regimens of the present disclosure range between about 5 mg to about 10 mg, about 6 mg to about 11 mg, about 7 mg to about 12 mg, about 8 mg to about 13 mg, about 9 mg to about 14 mg, about 9.5 mg to about 14.5 mg, and about 10 mg to about 15 mg.

In some embodiments, the CBG dosing regimens of the present disclosure range between about 9.5 mg to about 10 mg, about 9.5 mg to about 10.5 mg, and about 9.5 mg to about 11 mg.

The hemp extract dosing regimens of the serving sizes of the present disclosure include standardized dosages of THCV of about 1 mg, about 2 mg, about 3 mg, about 4 mg, about 4.5 mg, about 5 mg, about 5.5 mg, about 5.75 mg, about 5.8 mg, about 5.85 mg, about 5.9 mg, about 6 mg, about 6.1 mg, about 6.15 mg, about 6.2 mg, about 6.25 mg, about 6.5 mg, about 7 mg, about 8 mg, about 9 mg, 10 mg, and about 11 mg.

In some embodiments, the THCV dosing regimens of the present disclosure range between about 1 mg to about 7 mg, about 2 mg to about 8 mg, about 3 mg to about 9 mg, about 4 mg to about 9.5 mg, about 5 mg to about 10 mg, about 5.5 mg to about 10.5 mg, and about 6 mg to about 11 mg.

In some embodiments, the THCV dosing regimens of the present disclosure range between about 4.5 mg to about 5 mg; about 5 mg to about 5.5 mg, about 5.5 mg to about 6 mg, about 6 mg to about 6.5 mg, and about 6.5 mg to about 7 mg.

The hemp extract dosing regimens of the serving sizes of the present disclosure include standardized dosages of THC of about 1 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 8 mg, about 9 mg, about 9.5 mg, about 10 mg, about 10.5 mg, about 10.5 mg, about 11 mg, about 12 mg, about 13 mg, about 14 mg, and about 15 mg.

In some embodiments, the THC dosing regimens of the present disclosure range between about 1 mg to about 11 mg, about 2 mg to about 12 mg, about 3 mg to about 13 mg, and about 4 mg to about 15 mg.

In some embodiments, the THC dosing regimens of the present disclosure range between about 2 mg to about 3 mg, about 2.5 mg to about 3.5 mg, about 3 mg to about 4 mg, about 4 mg to about 5 mg; about 4.5 mg to about 5.5 mg, about 9 mg to about 10 mg, about 9.5 mg to about 10.5 mg, and about 10.5 to about 11.5.

The hemp extract dosing regimens of the serving sizes of the present disclosure include standardized dosages of CBN of about 1 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 8 mg, about 9 mg, about 9.5 mg, about 10 mg, about 10.5 mg, about 10.5 mg, about 11 mg, about 12 mg, about 13 mg, about 14 mg, and about 15 mg.

In some embodiments, the CBN dosing regimens of the present disclosure range between about 1 mg to about 11 mg, about 2 mg to about 12 mg, about 3 mg to about 13 mg, and about 4 mg to about 15 mg.

In some embodiments, the CBN dosing regimens of the present disclosure range between about 2 mg to about 3 mg, about 2.5 mg to about 3.5 mg, about 3 mg to about 4 mg, about 4 mg to about 5 mg; about 4.5 mg to about 5.5 mg, about 9 mg to about 10 mg, about 9.5 mg to about 10.5 mg, and about 10.5 to about 11.5.

The dosing regimens of the present disclosure includes dosages of Lion's mane powder of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, 750 mg, about 800 mg, about 850 mg, about 900 mg, about 950 mg, and about 1000 mg.

In some embodiments, the Lion's mane powder dosing regimens of the present disclosure range between about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, about 550 mg to about 600 mg, about 575 mg to about 625 mg, about 600 mg to about 650 mg, about 625 mg to about 675 mg, about 650 mg to about 700 mg, about 675 mg to about 725 mg, about 700 mg to about 750 mg, about 725 mg to about 775 mg, about 750 mg to about 800 mg, about 775 mg to about 825 mg, about 800 mg to about 850 mg, about 825 mg to about 875 mg, about 850 mg to about 900 mg, about 875 mg to about 925 mg, about 900 mg to about 950 mg, about 925 mg to about 975 mg, and about 950 mg to about 1000 mg.

In some embodiments, the Lion's mane powder dosing regimens of the present disclosure range between about 400 mg to about 600 mg, about 450 mg to about 650 mg, about 450 mg to about 550 mg, about 500 mg to about 600 mg, and about 550 mg to about 650 mg.

The dosing regimens of the present disclosure includes dosages of magnesium gluconate or magnesium oxide of about 100 mg, about 150 mg, about 200 mg, about 250 mg, about 300 mg, about 350 mg, about 400 mg, about 450 mg, and about 500 mg.

In some embodiments, the magnesium gluconate or magnesium oxide dosing regimens of the present disclosure range between about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, about 250 mg to about 300 mg, about 275 mg to about 325 mg, about 300 mg to about 350 mg, about 325 mg to about 375 mg, about 350 mg to about 400 mg, about 375 mg to about 425 mg, about 400 mg to about 450 mg, about 425 mg to about 475 mg, about 450 mg to about 500 mg, about 475 mg to about 525 mg, about 500 mg to about 550 mg, about 525 mg to about 575 mg, and about 550 mg to about 600 mg.

In some embodiments, the magnesium gluconate or magnesium oxide dosing regimens of the present disclosure range between about 200 mg to about 400 mg, about 250 mg to about 450 mg, about 250 mg to about 350 mg, about 300 mg to about 400 mg, and about 350 mg to about 450 mg.

The dosing regimens of the present disclosure includes dosages of L-Theanine of about 50 mg, about 75 mg, about 80 mg, about 85 mg, about 90 mg, about 95 mg, about 100 mg, about 110 mg, about 115 mg, about 120 mg, about 125 mg, about 130 mg, about 135 mg, about 140 mg, about 145 mg, about 150 mg, about 175 mg, about 200 mg, about 225 mg, and about 250 mg.

In some embodiments, the L-Theanine dosing regimens of the present disclosure range between about 75 mg to about 125 mg, about 100 mg to about 150 mg, about 125 mg to about 175 mg, about 150 mg to about 200 mg, about 175 mg to about 225 mg, about 200 mg to about 250 mg, about 225 mg to about 275 mg, and about 250 mg to about 300 mg.

In some embodiments, the L-Theanine dosing regimens of the present disclosure range between about 100 mg to about 150 mg, about 110 mg to about 140 mg, about 115 mg to about 135 mg, and about 120 mg to about 130 mg.

In some embodiments, the compositions as disclosed herein are typically prepared in a final liquid form for oral consumption of approximately 2 fluid ounces serving size (i.e., about 2 oz. or about 60 mL as a typical dose). In some instances, test users alternatively consumed approximately 1 fluid ounce per serving (i.e., about 1 oz. or about 30 mL, or about ½ of the typical serving size as disclosed herein) as a dose and other users preferred a double dose (i.e., about 4 fluid ounces or about 120 mL, or about twice the typical serving size as disclosed herein).

In some embodiments, the volume of oral suspension per dose of the present disclosure includes ranges between about 0.5 mL to about 1.0 mL, about 0.75 mL to about 1.25 mL, about 1.0 mL to about 1.5 mL, about 1.25 mL to about 1.75 mL, about 1.5 mL to about 2.0 mL, about 1.75 mL to about 2.25 mL, about 2.0 mL to about 2.5 mL, about 2.25 mL to about 2.75 mL, about 2.5 mL to about 3.0 mL, about 2.75 mL to about 3.25 mL, about 3.0 mL to about 3.5 mL, about 3.25 mL to about 3.75 mL, about 3.5 mL to about 4 mL, about 3.75 mL to about 4.25 mL, about 4.0 mL to about 4.5 mL, about 4.25 mL to about 4.75 mL, and about 4.5 mL to about 5 mL.

In some embodiments, the compositions as disclosed herein are typically prepared in softgel capsules for oral consumption with 2 softgels consumed at the same time (i.e., a typical dose is equal to 2 softgels). In some instances, test users alternatively consumed 1 softgel i.e., about ½ of the typical serving size as disclosed herein) or 4 softgels (i.e, about twice the typical serving size as disclosed herein).

In some embodiments, the number of softgels of the present disclosure consumed at one time can be 1 softgel, 2 softgels, 3 softgels, 4 softgels, 5 softgels, or 6 softgels.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

The following compositions are not recommended to be consumed by children (i.e., a person less than 12 years old). Adolescents (i.e., a person 12-21 years old) and adults (i.e., a person 22 years or older) should consult a healthcare profession before consuming these products if they are: (1) pregnant; (2) nursing; (3) have a medical condition; and/or (4) taking any medications. Users in the testing groups included men and women over the age of 21 who collectively ranged in age from their early 20's to their mid-80's.

Each of the following compositions provided herein include active ingredients as well as other, additional ingredients. The additional ingredients are not necessarily inert and can also have some activity but relatively less so than the ingredients identified as “active” herein.

In some embodiments the additional ingredients that can be added or substituted to the exemplary compositions provided herein include any disclosed herein elsewhere and also including but not limited to one or more of the following: water (e.g., distilled and/or filtered H₂O); one or more sweeteners (e.g., cane sugar (genus Saccharum), stevia extract (aka stevia liquid) (Stevia rebaudiana), tropical fruit punches, monk fruit extract (Siraitia grosvenorii)); natural flavorings (e.g., blueberry (genus Vaccinium), watermelon (Citrullus lanatus), mango (genus Mangifera), citric acid, tropical fruit punches, monk fruit extract (Siraitia grosvenorii), red pepper, black pepper, and/or chili pepper extracts (all from the genus Capsicum), vanilla fragrance oil (aka vanilla FO)); one or more emulsifiers (e.g., sunflower lecithin (genus Helianthus), soy lecithin (Glycine max)); additives for improving the bioavailability of poorly absorbed ingredients (e.g., VESIsorb®, including VESIsorb® 450); one or more preservatives (e.g., potassium sorbate, sodium benzoate, citric acid); and one or more anti-bitter agents or bitter blockers (e.g., ClearIQ™ from Myco Technology™).

In some embodiments of the present disclosure, these additional ingredients can serve two or more functions such as, for example, acting as an emulsifier and a surfactant; acting as a sweetener and a flavoring agent; and acting as a preservative and a flavoring agent.

The following examples utilize cannabis/hemp extract in a distillate form. The percentages of total cannabinoids and specific cannabinoids are standardized as indicated herein but the actual percentages will vary somewhat from batch to batch so there is some slight variation in any percentage value based on the actual potency of the oils used. Samples from one specific strain that was utilized had 70% D9 content which was taken into consideration for the following calculations where applicable. Samples that were utilized that were labeled “isolate” typically have a 98%+potency for the singular compound indicated, so that permits a 1:1 comparison for cannabinoid content.

Some of the alternate flavorings used in preparing the compositions include but are not limited to the following: (1) watermelon flavoring (water-based extract); (2) citrus (mandarin and clementine); (3) sweet heat (mango chili and medium heat chili extract); (4) tropical fruit punch (aka fruit punch); (5) blueberry; (6) cayenne pepper, and (7) cinnamon. Specific note of user reactions to these alternative flavorings are noted below where applicable.

### Example 1: Composition I—Improved Cognitive Function and Increased Ability to Focus

Composition I comprises an extract of Lion's mane mushroom (Hericium erinaceus) and an extract of the aerial parts of hemp (genus Cannabis). For an approximately 2 fluid ounce (i.e., about 2 oz. or about 60 mL) serving size of the composition, the amounts of mushroom extract and hemp extract per serving are about 500 mg and about 20 mg, respectively. The hemp extract is standardized to about 10 mg CBG, about 6 mg THCV, and about 3 mg THC. Based on Lion's mane mushroom supplied at 100% concentration, adding 0.84% to the composition will result in 504 mg/60 mL dose.

Table 1 provides one representative formula for a Composition I.

TABLE 1
Example formula for a Composition I.

| | | Approximate |
|---|---|---|
| | | Percentage (%) |
| Primary Function | Ingredient | by Weight |
| Diluent | Water | 93.0 |
| Emulsifier | VESIsorb ® | 0.4 |
| Co-Emulsifier | Sunflower Lecithin | 0.2 |
| Flavoring | Peppermint Oil | 0.1 |
| Flavoring | Stevia-liquid | 2.8 |
| Flavoring | Sucralose | 1.7 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | CBG Isolate | 0.02 |
| Active | THCV Isolate | 0.1 |
| Active | Lion's Mane | 0.8 |
| Active | D9 Distillate | 0.005 |
| Total | | ~100% |

Samples of Composition I were tested on various user groups orally administered about 2 fluid ounces once daily. The users scored their reactions to the samples on a scale of 1 (worst) to 10 (best). While this composition can be taken at any time, user input indicated it works well if taken in the morning (e.g., with breakfast), particularly before being active or doing professional work where attention to detail is essential. One user reported that one hour after consuming the product that they felt “focused and knocking out work without distraction and feel positive” [sic].

Users felt the effects of this composition within about 20 minutes of administration. Some users felt the effect within about 10 to about 15 minutes. One user felt that the peppermint version made the product “feel faster acting” and that the effect was almost immediate after administration.

Users on-average reported about two hours of improved cognitive function after consuming the product. One user reported the effect had worn off some after four hours but that person did not “feel a crash” and remained calm throughout the four hours.

Some users felt that an alternative version of Composition I with monk fruit extract and red pepper extract had “a nice flavor kick to awaken the senses.” At least one user felt that the mango chili version of this composition was a fun and uplifting option. One user preferred cayenne pepper and fruit punch-flavored version.

Users reported the composition was “easy to drink” and might be best served chilled or cold.

### Example 2: Composition II—Increased Energy Levels

Composition II comprises organic matcha powder (Camelia sinensis) and an extract of the aerial parts of hemp (genus Cannabis). For an approximately 2 fluid ounce (i.e., about 2 oz. or about 60 mL) serving size of the composition, the amounts of organic matcha powder and hemp extract per serving are about 2000 mg and about 16 mg, respectively. The hemp extract is standardized to about 10 mg CBG and about 6 mg THCV.

Table 2 provides one representative formula for a Composition II.

TABLE 2
Example formula for a Composition II.

| | | Approximate |
|---|---|---|
| | | Percentage (%) |
| Primary Function | Ingredient | by Weight |
| Diluent | Water | 88.2 |
| Emulsifier | VESIsorb ® | 1.8 |
| Co-Emulsifier | Sunflower Lecithin | 0.2 |
| Flavoring | Peppermint Oil | 0.5 |
| Flavoring | Stevia-liquid | 3.0 |
| Flavoring | Monk Fruit Extract | 2.0 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | CBG Isolate | 0.02 |
| Active | THCV Isolate | 0.1 |
| Active | Matcha Powder | 3.4 |
| Total | | ~100% |

Samples of Composition II were tested on various user groups orally administered about 2 fluid ounces once daily. The users scored their reactions to the samples on a scale of 1 (worst) to 10 (best). This composition can be taken at any time energy is needed, including in place of a shot of expresso, black tea, or coffee at breakfast. Also, this composition can be used later in the day to provide a “second wind on a tough day.”

Users scored this composition higher for tasting good as compared to most other matcha-based products. This composition provided users with a burst of energy within about 15-20 minutes of administration and provided about two hours of increased energy compared to not consuming the composition.

This product may be best served cold and an about 1 ounce serving size may be a good alternative (i.e., about ½ of the typical serving size). An alternative composition could include a higher dose of about 10 mg of THCV, which is about 167% more THCV by percentage than is in the typical serving size disclosed herein. Additional alternative composition could include adding mushroom extract and/or a spice, such as black pepper.

### Example 3. Composition III—Enhancing Clarity/Recreational

Composition III comprises an extract of the aerial parts of hemp (genus Cannabis). For an approximately 2 fluid ounce (i.e., about 2 oz. or about 60 mL) serving size of the composition, the amount of hemp extract per serving is 15 mg. The hemp extract is standardized to about 10 mg THC and about 5 mg THCV.

Table 3 provides representative Formula 1 for a Composition III.

TABLE 3
Example formula for Formula 1 for a Composition III.

| | | Approximate |
|---|---|---|
| | | Percentage (%) |
| Primary Function | Ingredient | by Weight |
| Diluent | Water | 84.0 |
| Emulsifier | VESIsorb ® | 7.7 |
| Co-Emulsifier | Sunflower Lecithin | 0.2 |
| Flavoring | Peppermint Oil | 0.2 |
| Flavoring | Stevia-liquid | 3.0 |
| Flavoring | Monk Fruit Extract | 2.0 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | D9 Distillate | 0.02 |
| Terpene | Beta Caryophyllene | 2.0 |
| Total | | ~100% |

Table 4 provides an alternative Formula 2 for a Composition III which includes a THCv isolate as an additional ingredient.

TABLE 4
Alternative example formula for Formula 2 for a Composition III.

| | | Approximate |
|---|---|---|
| | | Percentage (%) |
| Primary Function | Ingredient | by Weight |
| Diluent | Water | 84.0 |
| Emulsifier | VESIsorb ® | 7.7 |
| Co-Emulsifier | Sunflower Lecithin | 0.2 |
| Flavoring | Peppermint Oil | 0.2 |
| Flavoring | Stevia-liquid | 3.0 |
| Flavoring | Monk Fruit Extract | 2.0 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | D9 Distillate | 0.02 |
| Terpene | Beta Caryophyllene | 2.0 |
| Active | THCV Isolate | 0.01 |
| Total | | ~100% |

Samples of Formula 1 and 2 of Composition III were tested on various user groups orally administered about 2 fluid ounces once daily. The Beta Caryophyllene ingredient was removed from revised Formulas 1 and 2 of Composition III in later and final versions due to its unpleasant flavor impact as identified by the flavoring phase of Research & Development. The users scored their reactions to the samples on a scale of 1 (worst) to 10 (best). This composition is designed for recreational use and is directed to users with a higher tolerance to THC. This composition affected the user within about 20 minutes of administration, although some users did not realize it at first, and the affects lasted for about three hours.

The version of this composition with THCV reduced appetites. Users also reported that the THCV version had them feeling “loose and comfortable” and “smooth and chill” while it was also “not overwhelming.”

At least one user preferred the version of this composition with blueberry flavoring. Another user preferred the version with watermelon flavoring.

This composition can be produced in a larger sized product (e.g., an 8.4 ounce can with a pop top lid) that can be poured over ice and served like a cocktail to sip and enjoy in a more methodical way.

### Example 4. Composition IV—Boosting Productivity/Heightened Awareness

Composition IV comprises an extract of the aerial parts of hemp (genus Cannabis). For an approximately 2 fluid ounce (i.e., about 2 oz. or about 60 mL) serving size of the composition, the amount of hemp extract per serving is about 10 mg. The hemp extract is standardized to about 5 mg THC and about 5 mg THCV.

Table 5 provides representative Formula 1 for a Composition IV.

TABLE 5
Example formula for Formula 1 for a Composition IV.

| | | Approximate |
|---|---|---|
| | | Percentage (%) |
| Primary Function | Ingredient | by Weight |
| Diluent | Water | 85.1 |
| Emulsifier | VESIsorb ® | 7.2 |
| Co-Emulsifier | Sunflower Lecithin | 0.2 |
| Flavoring | Peppermint Oil | 0.1 |
| Flavoring | Stevia-liquid | 2.8 |
| Flavoring | Sucralose | 1.7 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | D9 Distillate | 0.01 |
| Terpene | Beta Caryophyllene | 2.0 |
| Total | | ~100% |

Table 6 provides an alternative Formula 2 for a Composition IV which includes a THCv isolate as an additional ingredient.

TABLE 6
Alternative example formula for Formula 2 for a Composition IV.

| | | Approximate |
|---|---|---|
| | | Percentage (%) |
| Primary Function | Ingredient | by Weight |
| Diluent | Water | 85.1 |
| Emulsifier | VESIsorb ® | 7.2 |
| Co-Emulsifier | Sunflower Lecithin | 0.2 |
| Flavoring | Peppermint Oil | 0.1 |
| Flavoring | Stevia-liquid | 2.8 |
| Flavoring | Sucralose | 1.7 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | D9 Distillate | 0.01 |
| Terpene | Beta Caryophyllene | 2.0 |
| Active | THCV Isolate | 0.001 |
| Flavoring | Vanilla FO | 0.01 |
| Total | | ~100% |

Samples of Formula 1 and 2 of Composition IV were tested on various user groups orally administered 2 fluid ounces once daily. The users scored their reactions to the samples on a scale of 1 (worst) to 10 (best). The Beta Caryophyllene ingredient was removed from revised Formulas 1 and 2 of Composition IV in later and final versions due to its unpleasant flavor impact as identified by the flavoring phase of Research & Development. This composition is designed for users who do not want to be too relaxed. Users found that this composition reduced food cravings and provided heightened awareness. Some users reported that they were hungry after consuming Formula 1 (without the THCV).

This composition affected the user within about 20 minutes of administration and the affects lasted for about two hours.

One user who consumed Formula 1 (no THCV) reported feeling the initial effects about 10 minutes following consumption, felt it stronger about 10 minutes later, and then felt it “peaking heavily after an hour of taking it.” One user who took Formula 1 still felt its effects about 3.5 hours after administration.

Users who consumed Formula 2 (with THCV) found its effects “very relaxing.” Users who were administered ½ of the normal dose (i.e., 1 fluid ounce or 30 mL) of Formula 2 reported that its effects lasted about 60-90 minutes.

At least one user preferred the version of the composition with an orange/clementine/citrus and vanilla flavoring mix.

Some iterations of Composition IV include monk fruit extract.

### Example 5. Composition V—Promoting Relaxation

Composition V comprises an extract of the aerial parts of hemp (genus Cannabis). For an approximately 2 fluid ounce (i.e., about 2 oz. or about 60 mL) serving size of the composition, the amounts of hemp extract per serving are about 8 mg. The hemp extract is standardized to about 5 mg CBN and about 3 mg THC.

Table 7 provides one representative formula for a Composition V.

TABLE 7
Example formula for a Composition V.

| | | Approximate |
|---|---|---|
| | | Percentage (%) |
| Primary Function | Ingredient | by Weight |
| Diluent | Water | 91.6 |
| Emulsifier | VESIsorb ® | 1.8 |
| Co-Emulsifier | Sunflower Lecithin | 0.2 |
| Flavoring | Peppermint Oil | 0.5 |
| Flavoring | Stevia-liquid | 3.0 |
| Flavoring | Monk Fruit Extract | 2.0 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | CBN Isolate | 0.01 |
| Active | D9 Distillate | 0.1 |
| Total | | ~100% |

Samples of Composition V were tested on various user groups orally administered about 2 fluid ounces once daily. Users started to feel more relaxed from about 15 to about 20 minutes after consuming it. The effect lasted for about three hours with no crash or noticeable decline. The users scored their reactions to the samples on a scale of 1 (worst) to 10 (best). This composition is well suited to being taken at any time when relaxation is desired, such as after a long day or lounging by the pool in the afternoon. Users reported it helped provide the ultimate chill mindset, helping the users slow down more than normal without feeling “stupid or silly.”

This composition also has a pineapple margarita flavor option which users noted had a very tropical smell, taste, and feel. At least one user preferred the tropical fruit punch flavor over the other versions. One small user group composed of 30-40-year-old women preferred the “tropical inspired” flavors.

### Example 6. Composition VI—Achieving Better Sleep Patterns

Composition VI comprises magnesium, L-Theanine, and an extract of the aerial parts of hemp (genus Cannabis). For an approximately 2 fluid ounce (i.e., about 2 oz. or about 60 mL) or 2 softgels serving size of the composition, the amounts of magnesium (e.g., magnesium gluconate for the liquid formula or magnesium oxide for the softgel formula), L-Theanine, and hemp extract are about 300 mg, 125 mg, and 10 mg, respectively. The hemp extract is standardized to about 10 mg of CBN.

Table 8 provides one representative formula for a Composition VI.

TABLE 8
Example formula for a Composition VI.

| | | Approximate |
|---|---|---|
| | | Percentage (%) |
| Primary Function | Ingredient | by Weight |
| Diluent | Water | 84.4 |
| Emulsifier | VESIsorb ® | 0.3 |
| Co-Emulsifier | Sunflower Lecithin | 0.2 |
| Flavoring | Peppermint Oil | 0.1 |
| Flavoring | Stevia-liquid | 2.8 |
| Flavoring | Sucralose | 1.7 |
| Preservative | Sodium Benzoate | 0.1 |
| Preservative | Potassium Sorbate | 0.1 |
| pH Adjuster | Citric Acid | 0.2 |
| Bitter Blocker | ClearIQ ™ | 0.5 |
| Active | Magnesium | 9.3 |
| | gluconate (liquid); | |
| | Magnesium oxide | |
| | (softgel) | |
| Active | CBN Isolate | 0.02 |
| Active | L-Theanine | 0.2 |
| Total | | ~100% |

Samples of Composition VI were tested on various user groups orally administered as about 2 fluid ounces or 2 softgels once daily. The users scored their reactions to the samples on a scale of 1 (worst) to 10 (best). This composition was a great sleep aid for all who consumed it. This composition is particularly well suited to the user that struggles with sleeping.

Consuming this composition has been shown to help promote getting to sleep and/or staying asleep when compared to not consuming it. The effects begin about 30 minutes after consumption. An alternative dosing schedule of consuming about 4 fluid ounces (about 4 oz. or about 120 mL; or 4 softgels) at one time may result in a faster reaction and quicker time falling asleep.

Focus group testing results have demonstrated that users had their best sleep scores after consuming this composition compared to not consuming it; and, that some users also experienced more vivid dreams compared to not consuming it. Users reported having a “sound sleep” and woke afterwards feeling more refreshed and rested compared to when they do not consume it. Users reported waking “with no fogginess or lull” and falling asleep quickly; and “did not feel any cannabinoid effects and woke up early without any grogginess.”

In some embodiments of the present disclosure, cinnamon flavoring and/or monk fruit extract is added to the composition in addition to or in replacement of the other flavorings and/or sweeteners listed in Table 8. At least one user felt that consuming the cinnamon flavored version before bedtime promoted a better, uninterrupted night's sleep than options without the cinnamon flavoring.

### Further Numbered Embodiments of the Disclosure

Other subject matter contemplated by the present disclosure is set out in the following numbered embodiments:
1. A composition for oral administration comprising about 500 mg of Lion's mane mushroom (Hericium erinaceus) and a hemp extract standardized to deliver about 10 mg of cannabigerol (CBG), about 6 mg of delta-9-tetrahydrocannabivarin (THCV), and about 3 mg delta-9-tetrahydocannabinol (THC).
2. A composition for oral administration comprising a hemp extract standardized to deliver about 10 mg cannabigerol (CBG) and about 6 mg of delta-9-tetrahydrocannabivarin (THCV).
3. A composition for oral administration comprising a hemp extract standardized to deliver about 5 mg of delta-9-tetrahydrocannabivarin (THCV) and about 5 mg or about 10 mg delta-9-tetrahydocannabinol (THC).
4. A composition for oral administration comprising a hemp extract standardized to deliver about 5 mg of cannabinol (CBN) and about 3 mg delta-9-tetrabydocannabinol (THC).

5. A composition for oral administration comprising about 300 mg of magnesium, about 125 mg L-Theanine, and a hemp extract standardized to deliver about 5 mg of cannabinol (CBN).
6. The composition of embodiment 5, wherein the magnesium is provided as magnesium gluconate and/or magnesium oxide.
7. The composition of any one of embodiments 1-6 further comprising one or more additional ingredients selected from the group consisting of a diluent, a flavoring, a preservative, a pH adjuster, a bitter blocker, and an emulsifier.
8. The composition of embodiment 7, wherein the diluent is water.
9. The composition of embodiment 7, wherein the one or more natural flavorings are selected from the group consisting of cinnamon, vanilla, monk fruit, peppermint, blueberry, citrus, chili, tropical fruit punch, and stevia.
10. The composition of embodiment 7, wherein the one or more preservatives are selected from the group consisting of sodium benzoate and potassium sorbate.
11. The composition of embodiment 7, wherein the pH adjuster is citric acid.
12. The composition of embodiment 7, wherein the one or more emulsifiers are selected from the group consisting of VESIsorb® and sunflower lecithin.
13. The composition of any one of embodiments 1-12, wherein the composition is the form of a liquid.
14. The composition of embodiment 13, wherein the liquid is provided in a 60 mL dose.
15. A method of administering a composition to an individual, wherein the method comprises the individual orally ingesting the liquid composition of embodiment 14.
16. The composition of any one of embodiments 1-12, wherein the composition is in the form of a soft gel capsule.
17. A method of administering a composition to an individual, wherein the method comprises the individual orally ingesting two soft gel capsules of embodiment 16.
18. A method of improving an individual's mind-body-balance comprising administering any one of the compositions of embodiments 1-14 or embodiment 16 to the individual.

### INCORPORATION BY REFERENCE

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### Other Publications

Lee J L C, Bertoglio L J, Guimarães F S, Stevenson C W. Cannabidiol regulation of emotion and emotional memory processing: relevance for treating anxiety-related and substance abuse disorders. Br J Pharmacol. 2017;174:3242-3256.
Peng J, Fan M, An C, et al. A narrative review of molecular mechanism and therapeutic effect of cannabidiol (CBD). Basic Clin Pharmacol Toxicol. 2022;130:439-456.
Pertwee R G. The diverse CB1 and CB2 receptor pharmacology of three plant cannabinoids: D9-tetrahydrocannabinol, cannabidiol and D9-tetrahydrocannabivarin. Br J Pharmacol. 2008;153:199-215.
Russo E B. Taming T H C: potential cannabissynergy and phytocannabinoid-terpenoid entourage effects. Br J Pharmacol. 2011;163(7):1344-64.
Russo, E. and G. W. Guy, A tale of two cannabinoids: the therapeutic rationale for combining tetrahydrocannabinol and cannabidiol. Med Hypotheses, 2006. 66(2): p. 234-46.

1. A composition for oral administration comprising about 500 mg of Lion's mane mushroom (Hericium erinaceus) and a hemp extract standardized to deliver about 10 mg of cannabigerol (CBG), about 6 mg of delta-9-tetrahydrocannabivarin (THCV), and about 3 mg delta-9-tetrabydocannabinol (THC).

2. A composition for oral administration comprising a hemp extract standardized to deliver about 10 mg cannabigerol (CBG) and about 6 mg of delta-9-tetrahydrocannabivarin (THCV).

3. (canceled)

4. (canceled)

5. A composition for oral administration comprising about 300 mg of magnesium, about 125 mg L-Theanine, and a hemp extract standardized to deliver about 5 mg of cannabinol (CBN).

6. The composition of claim 5, wherein the magnesium is provided as magnesium gluconate and/or magnesium oxide.

7. The composition of claim 1 further comprising one or more additional ingredients selected from the group consisting of a diluent, a flavoring, a preservative, a pH adjuster, a bitter blocker, and an emulsifier.

8. The composition of claim 7, wherein the diluent is water.

9. The composition of claim 7, wherein the one or more natural flavorings are selected from the group consisting of cinnamon, vanilla, monk fruit, peppermint, blueberry, citrus, chili, tropical fruit punch, and stevia.

10. The composition of claim 7, wherein the one or more preservatives are selected from the group consisting of sodium benzoate and potassium sorbate.

11. The composition of claim 7, wherein the pH adjuster is citric acid.

12. The composition of claim 7, wherein the one or more emulsifiers are selected from the group consisting of VESIsorb® and sunflower lecithin.

13. The composition of claim 1, wherein the composition is the form of a liquid.

14. The composition of claim 13, wherein the liquid is provided in a 60 mL dose.

15. A method of administering a composition to an individual, wherein the method comprises the individual orally ingesting the liquid composition of claim 14.

16. The composition of claim 1, wherein the composition is in the form of a soft gel capsule.

17. A method of administering a composition to an individual, wherein the method comprises the individual orally ingesting two soft gel capsules of claim 16.

18. A method of improving an individual's mind-body-balance comprising administering any one of the compositions of claim 1 to the individual.

19. The composition of claim 1 further comprising about 2,000 mg of (Camelia sinensis).
